(19) United States
(12) Patent Application Publication
(10) Pub. No.: US 2007/0166695 A1
(43) Pub. Date: Jul. 19, 2007

(54) Method for testing or screening for an enzyme of interest

(75) Inventor: Mads Eskelund Bjornvad, Frederiksberg (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/586,899

(22) PCT Filed: Feb. 9, 2005
(86) PCT No.: PCT/DK05/00084

§ 371 Date: Jul. 21, 2006

(30) Foreign Application Priority Data
Feb. 9, 2004 (DK) PA 2004 00192

(51) Int. Cl. C12Q 1/00

(52) U.S. Cl. 435/4

(57) ABSTRACT

The present invention relates to methods for testing or screening for an enzyme of interest.

### FIELD OF THE INVENTION

The present invention relates to methods for testing an enzyme of interest or screening a library of polypeptides by contacting it with a solid media comprising a substrate. In particular said methods may be performed by cultivating host cells expressing the enzyme of interest or library of polypeptides on or in a solid media.

### BACKGROUND OF THE INVENTION

Most assays directed towards testing or screening for a particular enzymatic activity are carried out in a liquid media.

Enzymes and libraries of polypeptides are often expressed by host cells, which are then tested or screened for expression of the enzyme of interest. Host cells are generally cultured in a liquid media or on a solid media, e.g. an agar plate.

Assays for testing the activity of an enzyme or screening for an enzyme of interest are often based on the use of a substrate which is able to change colour upon reaction with the enzyme or a substrate which is labelled with a dye which is then released upon reaction with the enzyme. The enzymatic activity may then be measured by measuring the colour formation.

When host cells are cultured on a solid media, assays for identification of host cells which express a particular enzymatic activity generally utilise a substrate for the enzyme which is capable of changing colour upon reaction with the enzyme or they utilise a substrate labelled with a dye which is released upon reaction between the substrate and the enzyme. Typically the reaction between the substrate and the enzyme results in the formation of a halo with a different colour than the rest of the solid media surrounding said host cells/colony of host cells expressing the particular enzyme, thereby making it possible to distinguish host cells expressing the enzyme from those not expressing the enzyme.

For example skim milk is often added to the solid media for detection of protease activity as proteases are capable of cleaving the proteins in the white skim milk and thereby make the skim milk colourless (i.e. host cells expressing the protease activity are detected by the presence of a colour-less halo surrounding them, also known as clearing zones).

Another example is detection of alpha-galactosidase by the release of p-nitrophenol (which is yellow) from p-nitrophenol-alpha-galactosid.

However, to increase the reliability and speed of the screening process for enzymatic activities there is a continuous need for improved assays for testing or screening for a particular enzymatic activity or expression of such enzymatic activity by a host cell.

Ligninolytic enzyme production by Polyporaceae from Lombok, Indonesia is disclosed by Risna R A and Suhirman (2002), Fungal Diversity, 9, 123-134.

WO 93/11249 discloses a method of screening for a DNA sequence coding for a protein of interest.

### SUMMARY OF THE INVENTION

In a first aspect the present invention relates to a method for testing an enzyme of interest or screening a library of polypeptides for an enzyme of interest comprising measuring the colour of a second dye, wherein the enzyme of interest or library of polypeptides has been contacted with a solid media in the presence of a first substrate, one or more other enzymes and a first dye, and wherein a product of the chemical reaction between an enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye.

In a second aspect the present invention relates to a method for testing a host cell or screening a library of host cells for expression of an enzyme of interest comprising measuring the colour of a second dye, wherein the host cell or library of host cells has been cultivated on or in a solid media in the presence of a first substrate, one or more other enzymes and a first dye, and wherein a product of the chemical reaction between the enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye.

In a third aspect the present invention relates to a method for testing an enzyme of interest or screening a library of polypeptides for an enzyme of interest comprising measuring the colour of a second dye, wherein the enzyme of interest or library of polypeptides has been contacted with a solid media in the presence of a first dye and a polymer, wherein the product of the chemical reaction between the first dye and the enzyme of interest is a second dye, and wherein the polymer is capable of binding to the second dye, and wherein the colour of the first dye is different from the colour of the second dye.

In a fourth aspect the present invention relates to a method for testing a host cell or a library of host cells for expression of an enzyme of interest comprising measuring the colour of a second dye, wherein the host cell or library of host cells has been cultivated on a solid media in the presence of a first dye and a polymer, wherein the product of the chemical reaction between the first dye and the enzyme of interest is a second dye, and wherein the polymer is capable of binding to the second dye, and wherein the colour of the first dye is different from the colour of the second dye.

In a fifth aspect the present invention relates to a method for testing or screening for an activity of a peroxidase (E.C. 1.11.1.7) comprising testing of screening for a change in the colour of Brilliant Blue by the presence of a host cell expressing the peroxidase, wherein the host cell has been cultivated on a solid media in the presence of Brilliant Blue and hydrogen peroxide.

In a sixth aspect the present invention relates to a method for testing or screening for an activity of a peroxidase (E.C. 1.11.1.7) comprising testing of screening for a change in the colour of Brilliant Blue by the presence of the peroxidase, wherein the peroxidase has been contacted with a solid media in the presence of Brilliant Blue and hydrogen peroxide.

### DEFINITIONS

In the context of the present invention, the term “E.C.” (Enzyme Class) refers to the internationally recognized enzyme classification system, Recommendations of the Nomenclature Committee of the International Union of Biochemistry and Molecular Biology, Academic Press, Inc. Unless otherwise indicated, it refers to the version of this recommendation from 1992. For those enzymes which are not present in the 1992-version it refers to the version of said recommendation found as the web-edition in January 2004, however for these enzymes the reaction that the enzyme catalyses is also indicated.

The term “substrate” or “substrate for an enzyme” is in the context of the present invention to be understood as a compound for which a chemical reaction converting the substrate into a product is catalysed by an enzyme.

The term “product” is in the context of the present invention to be understood as a compound produced by the chemical reaction converting a substrate into a product catalysed by an enzyme. The “substrate” and “product” of a chemical reaction catalysed by an enzyme are two chemically different compounds. However, a “product” of a chemical reaction catalysed by one enzyme may be a “substrate” of another chemical reaction catalysed by a different enzyme.

The term “nucleic acid sequence” is in the context of the present invention to be understood as a single-or double-stranded deoxyribonucleic acid (DNA) or ribonucleic acid (RNA) polynucleotide.

In the context of the present invention the term “a host cell expressing an enzyme” or “expression of an enzyme by a host cell” is to be understood as the transcription and translation of a nucleic acid sequence encoding an enzyme. Depending on the particular enzyme it may also include e.g. extracellular secretion of the enzyme by the host cells secretory pathway or transport to a intracellular organelle of the host cell. The enzyme may be e.g. the enzyme of interest.

The term “colour zone” used in relation to the host cells refers in the context of the present invention to a zone or area surrounding the host cell where a change in the colour of the solid media may be seen as a result of expression of the enzyme of interest.

The term “binding” or “bound” is in the context of the present invention to be understood as the establishment or the presence of any type of chemical and/or physical bond between two molecules, such as a covalent bond, a hydrogen bond, an electrostatic bond or ionic bond, or the attraction of molecules to each other by van der Waals forces.

### DETAILED DESCRIPTION OF THE INVENTION

### METHOD OF THE INVENTION

One embodiment of the present invention relates to a method for testing an enzyme of interest or screening a library of polypeptides for an enzyme of interest comprising measuring the colour of a second dye, wherein the enzyme of interest or library of polypeptides has been contacted with a solid media in the presence of a first substrate, one or more other enzymes and a first dye, and wherein a product of the chemical reaction between an enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye.

In particular a polymer capable of binding the second dye may further be present.

Said method may be performed as described in example 2 or 4, wherein the enzyme of interest or library of polypeptides is contacted with the first substrate by adding it to holes in the solid media, in this case agar, comprising the first substrate, the one or more other enzymes and the first dye. However, the present invention is not limited to this embodiment as other embodiments may be envisioned. Said method may be used to test the activity of a particular enzyme or it may be used to screen a library of polypeptides for an enzyme of interest. For example if the enzyme of interest or library of polypeptides is expressed and secreted by a host cell the enzyme of interest/library of polypeptides may be contacted with the solid media comprising the first substrate, the one or more other enzymes and the first dye, by adding the supernatant from the cultured host cells to said solid media. For enzymes or polypeptides which are not secreted but e.g. expressed intracellular the host cells may be lysed and then the supernatant of the lysed host cells may be contacted with said solid media. However, the enzyme of interest or library of polypeptides may also have been purified prior to contacting them with said solid media or they may have been prepared synthetically.

In another embodiment of the present invention the enzyme of interest or the library of polypeptides is expressed by a host cell which is cultivated in or on a solid media, whereby the enzyme of interest or the library of polypeptides is contacted with the solid media. Hence in another embodiment the present invention relates to a method for testing a host cell or screening a library of host cells for expression of an enzyme of interest comprising measuring the colour of a second dye, wherein the host cell or library of host cells has been cultivated on or in a solid media in the presence of a first substrate, one or more other enzymes and a first dye, and wherein a product of the chemical reaction between the enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye.

In the context of the present invention the term “in the presence” is to be understood as referring to that said compounds are able to interact with each other. In a particular embodiment the solid media comprises at least one of the compounds selected from the group consisting of one of more of the first substrate, one or more other enzymes and the first dye. Hence one or more of said compounds may in particular be incorporated in the solid media. However, other ways of ensuring that the enzyme of interest/library of polypeptide or the host cells/library of host cells are in the presence of the first substrate, one or more other enzymes and the first dye may be foreseen. For example one or more of said compounds may be applied to the solid media by e.g. spraying them onto the solid media.

Independently of whether the enzyme of interest/library of polypeptides is contacted with the solid media or expressed by a host cell which is cultivated in or on a solid media the following chemical reactions, schematically shown, may take place:
first substrate+enzyme of interest→(product+other enzyme)ₙ→product+other enzyme+first dye→second dye,
wherein “n” indicates the number of chemical reactions between a product and an other enzyme, wherein the product of each chemical reaction is a substrate for another other enzyme. Typically “n” may be 0, 1, 2, 3 or 4. More particularly “n” may be 0, 1 or 2. Above equation is only meant as an illustration showing the most relevant components of each chemical reaction, e.g. the enzymes involved are only shown as reactants and/or some of the products of a chemical reaction may not be shown.

In particular the chemical reaction between the other enzyme and the first dye may be a redox reaction; more particularly the other enzyme may catalyze oxidation of the first dye into a second dye.

In a particular embodiment said method(s) may be performed in the presence of a polymer capable of binding the second dye, e.g. a polymer capable of binding the second dye may be present in the solid media. As described below the inventor of the present invention has found that the presence of said polymer appears to fixate the formed second dye in the solid media so that it does not diffuse. Diffusion of the formed second dye is a well-known problem for many methods related to testing or screening for an enzymatic activity.

Both methods may comprise the steps of a) contacting the enzyme of interest/library of polypeptides with a solid media in the presence of a first substrate, one or more other enzymes and a first dye, and wherein a product of the chemical reaction between an enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye and b) measuring the colour of the second dye. The activity of the enzyme of interest is indirectly measured by measuring the colour of the second dye as the second dye is produced if the enzyme of interest is present. If the enzyme of interest/library of polypeptides is expressed by a host cell step a) may be performed by cultivating the host cell on or in the solid media. Thus said method may then comprise the following steps:
a) cultivating a host cell expressing the enzyme of interest or a library of host cells expressing a library of polypeptides on or in a solid media in the presence of a first substrate, one or more other enzymes and a first dye, wherein a product of the chemical reaction between the enzyme of interest and the first substrate is a substrate for one of the other enzymes, and wherein the first dye is a substrate for one of the other enzymes, and wherein the product of the chemical reaction between the first dye and one of the other enzymes is a second dye, and wherein the colour of the first dye is different from the colour of the second dye.
b) measuring the colour of the second dye

Another embodiment of the present invention relates to a method for testing an enzyme of interest or screening a library of polypeptides for an enzyme of interest comprising measuring the colour of a second dye, wherein the enzyme of interest or library of polypeptides has been contacted with a solid media in the presence of a first dye and a polymer, and wherein the product of the chemical reaction between the first dye and the enzyme of interest is a second dye, and wherein the polymer is capable of binding to the second dye, and wherein the colour of the first dye is different from the colour of the second dye.

The enzyme of interest or the library of polypeptides may be expressed by a host cell which is cultivated on or in a solid media. Hence the present invention also relates to a method for testing a host cell or a library of host cells for expression of an enzyme of interest comprising measuring the colour of a second dye, wherein the host cell or library of host cells has been cultivated on or in a solid media in the presence of a first dye and a polymer, wherein the product of the chemical reaction between the first dye and the enzyme of interest is a second dye, and wherein the polymer is capable of binding to the second dye, and wherein the colour of the first dye is different from the colour of the second dye.

In particular both methods may comprise the steps of a) contacting the enzyme of interest or the library of polypeptides with a solid media in the presence of a first dye and a polymer, wherein the product of the chemical reaction between the first dye and the enzyme of interest is a second dye, and wherein the polymer is capable of binding to the second dye, and wherein the colour of the first dye is different from the colour of the second dye and b) measuring the colour of the second dye. If the enzyme of interest/library of polypeptides is expressed by a host cell step a) may be performed by cultivating the host cell on or in the solid media. Thus said method may then comprise the following steps:
a) cultivating a host cell expressing the enzyme or a library of host cells expressing a library of polypeptides on or in a solid media in the presence of a first dye and a polymer, wherein the product of the chemical reaction between the first dye and the enzyme of interest is a second dye, and wherein the polymer is capable of binding to the second dye, and wherein the colour of the first dye is different from the colour of the second dye.
b) measuring the colour of the second dye

The inventor of the present invention have found that the presence of a polymer capable of binding the second dye can reduce the diffusion of the second dye in the solid media and thereby ease detection of an enzyme of interest and/or a host cell expressing an enzyme of interest. Thus the use of a polymer may aid in distinguishing an enzyme of interest from other polypeptides, e.g. distinguishing between host cells expressing an enzyme of interest from host cells not expressing said enzyme. The presence of the polymer is particularly advantageous if the colour zone surrounding the enzyme of interest or the host cell does not have a well-defined border.

In yet another embodiment the present invention relates to a method for testing or screening for an activity of a peroxidase (E.C. 1.11.1.7) comprising testing of screening for a change in the colour of Brilliant Blue by the presence of the peroxidase, wherein the peroxidase has been contacted with a solid media comprising Brilliant Blue and hydrogen peroxide.

In particular the peroxidase may be expressed by a host cell. Thus in particular the present invention relates to a method for testing or screening for an activity of a peroxidase (E.C. 1.11.1.7) comprising testing of screening for a change in the colour of Brilliant Blue by the presence of a host cell expressing the peroxidase, wherein the host cell has been cultivated on or in a solid media in the presence of Brilliant Blue and hydrogen peroxide. The inventor of the present invention has found that Brilliant Blue is particularly useful to detect peroxidase activity when a peroxidase is contacted with a solid media in the presence of Brilliant Blue and hydrogen peroxide.

The methods of the present invention may be used to test the enzymatic activity of one enzyme or it may be used to screen for an enzymatic activity. In particular the methods of the present invention may be used to screen a library of host cells for expression of a particular enzymatic activity. For example said methods may be used to screen for mutants of known enzymes or to screen one or more organism for expression of a particular enzymatic activity. This may performed by creation of a library of nucleic acid sequences encoding different mutants of a known enzyme or a library of nucleic acid sequences encoding different polypeptides expressed by a particular organism and subsequently introducing said library into a host cell.

The methods of the present invention have several advantages. One advantage is that it is not necessary to have a substrate for the enzyme of interest which is capable of changing colour upon reaction with the enzyme of interest. Furthermore, it is not necessary with the present method to use a substrate labelled with a dye. Hence it is possible to test the activity of the enzyme of interest with a substrate which it is intended to interact with in a real application. One advantage of the methods of the present invention is that it is possible to test enzymatic activities towards substrate which may be difficult to solubilise and/or the conditions at which is soluble may be incompatible with an expression host cell.

An advantage of testing the enzyme or screening a library of polypeptides directly with a method of the present invention instead of e.g. testing or screening host cells for expression of an enzyme of interest is that assay conditions do not need to be compatible with the host cell.

Enzyme of Interest

In one embodiment of the present invention the enzyme of interest may be any enzyme for which a product of the chemical reaction between the enzyme of interest and a substrate (i.e. a first substrate) is a substrate for another enzyme.

For example the enzyme of interest may in particular be a glucosidase (E.C. 3.2.1), such as a glucan 1,4-alpha-glucosidase (also known as glucoamylase) (E.C. 3.2.1.3), e.g. the G1 or G2 form of the glycoamylase from Aspergillus niger (SWISSPROT:AMYG_ASPNG Prim. accession #P04064 and reference “Glucoamylases G1 and G2 from Aspergillus niger are synthesized from two different but closely related mRNAs”, Boel, E et al. (1984), EMBO J. 3:1097), or it may be the glycoamylase from Talaromyces emersonii (WO9928448). Glucoamylases are sometimes described as amyloglucosidase which may shorten to AMG. It may also be an alpha-amylase (E.C. 3.2.1.1), such as a Bacillus alpha-amylase (often referred to as “Termamyl-like alpha-amylases”). Well-known Termamyl-like alpha-amylases include alpha-amylase derived from a strain of B. licheniformis (commercially available as Termamyl), B. amyloliquefaciens, and B. stearothermophilus alpha-amylase. Other Termamyl-like alpha-amylases include alpha-amylase derived from a strain of the Bacillus sp. NCIB 12289, NCIB 12512, NCIB 12513 or DSM 9375, all of which are described in detail in WO95/26397, and the alpha-amylase described by Tsukamoto et al., Biochemical and Biophysical Research Communications, 151 (1988), pp. 25-31. In the context of the present invention a Termamyl-like alpha-amylase is an alpha-amylase as defined in WO99/19467 on page 3, line 18 to page 6, line 27. Contemplated variants and hybrids are described in WO96/23874, WO97/41213, and WO99/19467. Specifically contemplated is a recombinant B.stearothermophilus alpha-amylase variant with the mutations: I181*+G182*+N193F. Bacillus alpha-amylases may be added in effective amounts well known to the person skilled in the art.

It may also be a beta-amylase (E.C. 3.2.1.2), such as a beta-amylase obtained from wheat or barley, e.g. Novozym WBA®, or a glucan 1,4-alpha-maltohydrolase (E.C. 3.2.1.133), i.e. an exo-acting maltogenic alpha-amylase as described in WO 9104669 or WO 9943794 of which an example is Novamyl® (product of Novozymes A/S).

The enzyme of interest may also be a pectinesterase (E.C. 3.1.1.11), a cellulase (E.C. 3.2.1.4), a beta-glucosidase (E.C. 3.2.1.21), an alpha-galactosidase (E.C. 3.2.1.22), a glucan 1,3-beta-glucosidase (E.C. 3.2.1.58), a cellulose 1,4-beta-cellobiosidase (E.C. 3.2.1.91), a lactase (E.C. 3.2.1.108), a beta-galactofuranosidase (the web-edition from January 2004: E.C. 3.2.1.146; enzymes catalyzing the reaction of: Hydrolysis of terminal non-reducing b-D-galactofuranosides, releasing galactose), a carboxypeptidase A (E.C. 3.4.17.1) or a aldose 1-epimerase (E.C. 5.1.3.3).

In another particular embodiment the enzyme of interest may be an enzyme capable of producing hydrogen peroxide upon reaction with a substrate for the enzyme. In particular it may be an oxidase, such as a malate oxidase (E.C. 1.1.3.3), a glucose oxidase (E.C. 1.1.3.4), e.g. glucose oxidase derived from Aspergillus niger, hexose oxidase (E.C. 1.1.3.5), cholesterol oxidase (E.C. 1.1.3.6), aryl-alcohol oxidase (E.C. 1.1.3.7), L-gulonolactone oxidase (E.C. 1.1.3.8), galactose oxidase (E.C. 1.1.3.9), pyranose oxidase (E.C. 1.1.3.10), L-sorbose oxidase (E.C. 1.1.3.11), pyridoxine oxidase (E.C. 1.1.3.12), alcohol oxidase (E.C. 1.1.3.13), (S)-2-hydroxy-acid oxidase (E.C. 1.1.3.15), ecdysone oxidase (E.C. 1.1.3.16), choline oxidase (E.C. 1.1.3.17), secondary-alcohol oxidase (E.C. 1.1.3.18), 4-hydroxymandalate oxidase (E.C. 1.1.3.19), glycerol-3-phosphate oxidase (E.C. 1.1.3.21), xanthine oxidase (E.C. 1.1.3.22), thiamine oxidase (E.C. 1.1.3.23), L-galactonolactone oxidase (E.C. 1.1.3.24), cellobiose oxidase (E.C. 1.1.3.25), hydroxypythanate oxidase (E.C. 1.1.3.27), N-acylhexosamine oxidase (E.C. 1.1.3.29), polyvinyl-alcohol oxidase (E.C. 1.1.3.30), D-arabino-1,4-lactone oxidase (the web-edition from January 2004: E.C. 1.1.3.37; enzymes catalyzing the reaction of: D-arabinono-1,4-lactone+O2→D-erythro-ascorbate+H2O2), vanillyl-alcohol oxidase (the web-edition from January 2004: E.C. 1.1.3.38; enzymes catalyzing the reaction of: vanillyl alcohol+O2→vanillin+H2O2), nucleoside oxidase (H2O2—forming) (the web-edition from January 2004: E.C. 1.1.3.39; enzymes catalyzing the reaction of: adenosine+2O2→9-riburonosyladenine+2 H2O2), D-mannitol oxidase (the web-edition from January 2004: E.C. 1.1.3.40; enzymes catalyzing the reaction of: mannitol+O2→mannose+H2O2) or xylitol oxidase (the web-edition from January 2004: E.C. 1.1.3.41; enzymes catalyzing the reaction of: xylitol+O2→xylose+H2O2).

Other examples of relevant oxidases include, but are not limited to, aldehyde oxidase (E.C. 1.2.3.1), Pyruvate oxidase (E.C.1.2.3.3), Oxalate oxidase (E.C. 1.2.3.4), Glyoxylate oxidase (E.C. 1.2.3.5), Pyruvate oxidase (CoA-acetylating) (E.C. 1.2.3.6), Aryl-aldehyde oxidase (E.C. 1.2.3.9), Retinal oxidase (E.C. 1.2.3.11), Dihydroorotate oxidase (E.C. 1.3.3.1), Lathosterol oxidase (E.C.1.3.3.2), Acyl-CoA oxidase (E.C. 1.3.3.6), Dihydrouracil oxidase (E.C.1.3.3.7), Tetrahydroberberine oxidase (E.C.1.3.3.8), D-aspartate oxidase (E.C.1.4.3.1), L-amino acid oxidase (E.C.1.4.3.2), D-amino acid oxidase (E.C.1.4.3.3), Amine oxidase (flavin-containing) (E.C. 1.4.3.4), Pyridoxamine-phosphate oxidase (E.C. 1.4.3.5), Amine oxidase (copper-containing) (E.C.1.4.3.6), D-glutamate oxidase (E.C.1.4.3.7), Ethanolamine oxidase (E.C.1.4.3.8), Putrescine oxidase (E.C. 1.4.3.10), L-glutamate oxidase (E.C.1.4.3.11), Cyclo-hexylamine oxidase (E.C.1.4.3.12), Protein-lysine 6-oxidase (E.C.1.4.3.13), L-lysine oxidase (E.C.1.4.3.14), D-glutamate(D-aspartate) oxidase (E.C.1.4.3.15), L-aspartate oxidase (E.C.1.4.3.16), Glycine oxidase (the web-edition from January 2004: E.C.1.4.3.19; enzymes capable of catalyzing one of the following reactions: (1) glycine+H2O+O2→glyoxylate+NH3+H2O2; (2) D-alanine+H2O+O2→pyruvate+NH3+H2O2; (3) sarcosine+H2O+O2→glyoxylate+methylamine+H2O2; (4) N-ethylglycine+H2O+O2→glyoxylate+ethyl-amine+H2O2), Sarcosine oxidase (E.C. 1.5.3.1), N-methyl-L-amino-acid oxidase (E.C.1.5.3.2), N(6)-methyl-lysine oxidase (E.C.1.5.3.4), (S)-6-hydroxynicotine oxidase (E.C.1.5.3.5), (R)-6-hydroxynicotine oxidase (E.C.1.5.3.6), L-pipecolate oxidase (E.C.1.5.3.7), Dimethylglycine oxidase (E.C.1.5.3.10), Polyamine oxidase (E.C.1.5.3.11), Dihydrobenzophe-nanthridine oxidase (the web-edition from January 2004: E.C.1.5.3.12; enzymes capable of catalyzing one of the following reactions: (1) dihydrosanguinarine+O2→sanguinarine+H2O2; (2) dihydrochelirubine+O2→chelirubine+H2O2; (3) dihydromacarpine+O2→macarpine+H2O2), NADPH oxidase (the web-edition from January 2004: E.C.1.6.3.1; enzymes capable of catalyzing the reaction of: NAD(P)H⁺H⁺+O2→NAD(P)⁺+H2O2).

In another embodiment of the present invention the enzyme of interest may be any enzyme for which a product of the chemical reaction between the enzyme of interest and a first dye is a second dye, wherein the colour of the first dye is different from the colour of the second dye. In particular the enzyme of interest for this method may be a peroxidase (E.C. 1.11.1.7). Peroxidases are enzymes capable of catalyzing the oxidation, by hydrogen peroxide, of a number of substrates such as ascorbate, cytochrome C and the leuco form of many dyes. A representative reaction is shown below:
Peroxidase+H2O2+DH2→2H2O+D,
wherein DH2=leuco dye and D=dye

In particular the peroxidase may be Horseradish peroxidase (HRP) which exists in the form of several isozymes, all containing heme as the prosthetic group. The enzyme has a molecular weight of approximately 40,000. For example it may be the Horseradish peroxidase from Sigma-Aldrich, product number P8125.

The ability of peroxidase to catalyze the oxidation of a number of organic compounds by hydrogen peroxide, resulting in formation of colored end-products, is utilized in several methods of determination of glucose and galactose in biological fluids.

The enzyme of interest may be naturally expressed by a host cell or it may be an enzyme which a host cell has been manipulated to express, i.e. by introduction of a nucleic acid sequence encoding the enzyme of interest into a host cell. Thus the nucleic acid sequence encoding the enzyme of interest, i.e. the nucleic acid sequence of interest may be native or foreign to the host cell. In this context the term “native” refers to a nucleic acid sequence which is naturally present in the genome of the host cell, while the term “foreign” refers to a nucleic acid sequence which is not normally present in the host cell of the invention, i.e. a nucleic acid sequence which has been introduced into the genome of the host cell.

Library of Polypeptides

The present invention also relates to a method for screening a library of polypeptides or a library of host cells for an enzyme of interest. In the context of the present invention the term “library of polypeptides” and “library of host cells” is to be understood as a collection of at least two different polypeptides and a collection of at least two host cells expressing at least two different polypeptides; i.e. at least two polypeptides which differ at one or more amino acid positions, e.g. the number of amino acids in the polypeptides may be different and/or the amino acid(s) at a particular position may be different. Typically a library of host cells consist of host cells which are more or less identical, e.g. same species, except for the expression of a different polypeptide.

Typically, the library of polypeptides may be prepared by introducing a library of nucleic acid sequences encoding the library of polypeptides into a host cell capable of expressing the polypeptides. Due to the genetic degeneracy the number of different nucleic acid sequences in said library may be higher than the number of different polypeptides which are actually expressed by the host cell.

In particular said library of nucleic acid sequences may encode variants of a parent enzyme; i.e. polypeptides which differ at, at least one amino acid position compared to a parent enzyme. Thus the screening method may be used to screen for variants of a parent enzyme. Such variants may be produced by e.g. random mutagenesis or site-directed mutagenesis of a parent enzyme or by other methods known to a person skilled in the art. Thus in a particular embodiment the library of polypeptides may be a library of variants of parent enzyme. Examples of suitable parent enzymes include but are not limited to those mentioned above in the section of enzymes. In particular the parent enzyme may be a lipolytic enzyme e.g. a lipase from Humicola, e.g. H. lanuginosa, or Pseudomonas or Bacillus.

In another embodiment said library of nucleic acid sequences may encode polypeptides derived from one or a number of different organisms. Thus the method may be used to screen one or a number of different organisms for expression of an enzyme activity of interest.

In another embodiment the library of polypeptides may be prepared by synthesizing the polypeptides.

An advantage of using the method of the present invention to screen, e.g. a diversified library is that such libraries often contain a substantially high proportion of inactive variants (e.g. inactive enzyme variants). It is therefore desirable to enrich the proportion of active clones before initiating a high-through-put screen based on e.g. microtiter plates or other encapsulation techniques.

Methods for preparing a library of nucleic acid sequences, introducing it into a host cell, expressing the polypeptides encoded by said library of polypeptides in the host cells and methods for synthesizing polypeptides are well known to a person skilled in the art and may e.g. be found in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “Molecular Biological Methods for Bacillus”, John Wiley and Sons, (1990); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “Transcription And Translation”, B. D. Hames & S. J. Higgins, eds. (1984); “Animal Cell Culture”, R. I. Freshney, ed. (1986); “Immobilized Cells And Enzymes”, IRL Press, (1986); “A Practical Guide To Molecular Cloning”, B. Perbal, (1984).

As described previously the nucleic acid sequence of interest may be a single-or double-stranded DNA or RNA polynucleotide. The enzyme of interest may in particular be encoded by a genomic DNA or a cDNA sequence.

The enzyme of interest may derive from any cell, including but not limited to one of those described as host cells below.

First Substrate

The first substrate of the present invention may be any compound which is a substrate for the enzyme of interest. Thus the choice of the first substrate depends on the enzyme of interest. Substrates for different enzymes are known to a person skilled in the art. In one embodiment of the present invention the product of the reaction between the enzyme of interest and the first substrate should be a substrate for one of the other enzyme(s).

For example if the enzyme of interest in this embodiment is a glucoamylase (E.C. 3.2.1.3) the first substrate may for example be selected from the group consisting of but not limited to: maltose, maltodextrin, starch, e.g. potato starch.

If the enzyme of interest is an alpha-amylase (E.C. 3.2.1.1) or a beta-amylase (E.C.3.2.1.2) or a glucan 1,4-alpha-maltohydrolase (E.C. 3.2.1.133) the first substrate may be maltotriose.

If the enzyme of interest is a cellulase (E.C. 3.2.1.4) the first substrate may be cellulose.

If the enzyme of interest is a glucose oxidase (E.C. 1.1.3.4.) the first substrate may be beta-D-glucose.

If the enzyme of interest is a galactose oxidase (E.C. 1.1.3.9) the first substrate may be D-galactose.

If the enzyme of interest is an alcohol oxidase (E.C. 1.1.3.13) the first substrate may be a primary alcohol.

If the enzyme of interest is a L-amino acid oxidase (E.C. 1.4.3.2) the first substrate may be an L-amino acid.

In another embodiment of the present invention the enzyme of interest may be any enzyme for which a product of the chemical reaction between the enzyme of interest and a first dye is a second dye, wherein the colour of the first dye is different from the colour of the second dye. Thus if the enzyme of interest is peroxidase (E.C. 1.11.1.7) the first substrate may be a first dye, in particular it may be a compound which is capable of being oxidized by peroxidase into a second dye. Examples of such first dyes are given below.

Other Enzymes

One embodiment of the present invention relates to a method for detection of the activity of an enzyme comprising using a first substrate, one or more other enzymes and a first dye. The principle behind this method is that a product produced by the conversion of the first substrate to a product catalysed by the enzyme of interest is a substrate for one of the other enzymes. The product produced by the conversion of this substrate catalysed by one of the other enzymes may then again be a substrate for one of the other enzymes. In this way a chain of chemical reactions converting a substrate to a product catalysed by an enzyme is created. Thus in principle the method utilizes a cascade of chemical reactions catalyzed by enzymes leading from the first chemical reaction between the enzyme of interest and the first substrate to the last chemical reaction between one of the other enzymes and the first dye. As previously shown this may be shown schematically in the following way:
first substrate+enzyme of interest→(product+other enzyme)ₙ→product+other enzyme+first dye→second dye,
wherein “n” indicates the number of chemical reactions between a product and an other enzyme, wherein the product of each chemical reaction is a substrate for another other enzyme. Typically “n” may be 0, 1, 2, 3 or 4. More particularly “n” may be 0, 1 or 2.

The choice of the other enzymes depends on the particular enzyme of interest and the first substrate. However, in a particular embodiment the other enzymes may comprise at least a peroxidase.

In another particular embodiment the other enzymes may comprise at least an enzyme capable of producing hydrogen peroxide upon reaction with a substrate for the enzyme, and a peroxidase. In this case the first dye may particularly be a compound capable of being oxidized to a second dye. Examples of peroxidase of particular interest are those described above as enzyme of interest.

For example, for testing or screening for a glucoamylase activity the enzyme of interest may be a glucoamylase (E.C. 3.2.1.3), the first substrate may be maltose, maltodextrin or a starch, e.g. potato starch, the other enzymes may be a glucose oxidase (E.C. 1.1.3.4) and a peroxidase (E.C. 1.11.1.7) and the first dye may be a compound capable of being oxidized by a peroxidase, e.g. Brilliant Blue. Thus in this case the following cascade of enzyme catalysed chemical reactions may take place:
Maltose+glucoamylase→Glucose+Glucose Oxidase→H2O2+first dye (red)+Peroxidase→second dye (ox)
wherein the term “(red)” and “(ox)” refers to the reduced and oxidized forms of the same compound.

Or for testing or screening for a cellulase activity the enzyme of interest may be a cellulase (E.C.3.2.1.4), the first substrate may be cellulose, the other enzymes may be cellobiose oxidase (E.C. 1.1.3.25) and a peroxidase (E.C. 1.11.1.7), and the first dye may be a compound capable of being oxidized by a peroxidase. Thus in this case the following cascade of enzyme catalysed chemical reactions may take place:
Cellulose+cellulase→cellobiose+cellobiose oxidase→H2O2+first dye (red)+Peroxidase→second dye (ox)
wherein the term “(red)” and “(ox)” refers to the reduced and oxidized forms of the same compound.

Another way of testing or screening for a cellulase activity include using the same components as described above but with the exception of using a beta-glucosidase (E.C. 3.2.1.21) and a glucose oxidase (E.C. 1.1.3.4) instead of a cellobiose oxidase as a other enzyme(s). Thus in this case the following reaction takes place:
Cellulose+cellulase→cellobiose+beta-glucosidase→glucose+glucose oxidase→H2O2+first dye (red)+Peroxidase→second dye (ox)
wherein the term “(red)” and “(ox)” refers to the reduced and oxidized forms of the same compound.

Similarly, for testing or screening for a beta-glucosidase activity the above reaction may be used with a beta-glucosidase as enzyme of interest (E.C. 3.2.1.21), cellobiose as the first substrate, a glucose oxidase (E.C. 1.1.3.4) and a peroxidase (E.C. 1.11.1.7) as the other enzymes and a compound capable of being oxidized by a peroxidase as the first dye. Thus in this case the following cascade of enzyme catalysed chemical reactions may take place:
Cellobiose+beta-glucosidase→glucose+glucose oxidase→H2O2+first dye (red) +Peroxidase→second dye (ox)
wherein the term “(red)” and “(ox)” refers to the reduced and oxidized forms of the same compound.

If the enzyme of interest is a pectinesterase (E.C. 3.1.1.11), the first substrate may e.g. be pectin, the other enzymes may be an alcohol oxidase (E.C. 1.1.3.13) and a peroxidase (E.C. 1.11.1.7), and the first dye may be a compound capable of being oxidized by a peroxidase. Thus, in this case the following cascade of enzyme catalysed chemical reactions may take place:
Pectin+pectinesterase→methanol+alcohol oxidase→H2O2+first dye (red)+Peroxidase→second dye (ox),
wherein the term “(red)” and “(ox)” refers to the reduced and oxidized forms of the same compound.

Or if the enzyme of interest is an alpha-galactosidase (E.C. 3.2.1.22), the first substrate may be melibiose, the other enzymes may be a glucose oxidase (E.C. 1.1.3.4) and a peroxidase (E.C. 1.11.1.7) or the other enzymes may be a galactose oxidase (E.C. 1.1.3.9) and a peroxidase (E.C. 1.11.1.7), and the first dye may be a compound capable of being oxidized by a peroxidase. Thus in this case one of the following cascades of enzyme catalysed chemical reactions may take place:
Melibiose+alpha-galactosidase→glucose+galactose+glucose oxidase→H2O2+first dye (red)+Peroxidase→second dye (ox),
or
Melibiose+alpha-galactosidase→glucose+galactose+galatose oxidase→H2O2+first dye (red)+Peroxidase→second dye (ox),
wherein the term “(red)” and “(ox)” refers to the reduced and oxidized forms of the same compound.

If the enzyme of interest is a cellulose 1,4-beta-cellobiosidase (E.C. 3.2.1.91), the first substrate may be cellulose or cellotetraose, the other enzymes may be a cellobiose oxidase (E.C. 1.1.3.25) and a peroxidase (E.C. 1.11.1.7), and the first dye may be a compound capable of being oxidized by a peroxidase. Thus with cellulose as the first substrate the following cascade of enzyme catalysed chemical reactions may take place:
Cellulose+cellulose 1,4-beta-cellobiosidase→Cellobiose+cellobiose oxidase→H2O2+first dye (red)+Peroxidase→second dye (ox)
wherein the term “(red)” and “(ox)” refers to the reduced and oxidized forms of the same compound.

Another way for testing or screening for the activity of a cellulose 1,4-beta-cellobiosidase (E.C. 3.2.1.91) include using the same components as described above but with the exception that a beta-glucosidase (E.C. 3.2.1.21), a glucose oxidase (E.C. 1.1.3.4) and a peroxidase (E.C. 1.11.1.7) are used as the other enzyme instead of the cellobiose oxidase and the peroxidase. Thus with cellulose as the first substrate the following cascade of enzyme catalysed chemical reactions may take place:
Cellulose+cellulose 1,4-beta-cellobiosidase→Cellobiose+beta-glucosidase→glucose+glucose oxidase→H2O2+first dye (red)+Peroxidase→second dye (ox)
wherein the term “(red)” and “(ox)” refers to the reduced and oxidized forms of the same compound.

If the enzyme of interest is a lactase (E.C. 3.2.1.108), the first substrate may be lactose, the other enzymes may be a glucose oxidase (E.C. 1.1.3.4) and a peroxidase (E.C. 1.11.1.7) or the other enzymes may be a galactose oxidase (E.C. 1.1.3.9) and a peroxidase (E.C. 1.11.1.7), and the first dye may be a compound capable of being oxidized by a peroxidase. Thus in this case one of the following cascades of enzyme catalysed chemical reactions may take place:
Lactose+lactase→glucose+galactose+glucose oxidase→H2O2+first dye (red)+Peroxidase→second dye (ox),
or
Lactose+lactase→glucose+galactose+galatose oxidase→H2O2+first dye (red)+Peroxidase→second dye (ox),
wherein the term “(red)” and “(ox)” refers to the reduced and oxidized forms of the same compound.

If the enzyme of interest is a beta-galactofuranosidase (the web-edition from January 2004: E.C. 3.2.1.146), the first substrate may be beta-D-galactofuranoside, the other enzymes may be a galactose oxidase (E.C. 1.1.3.9) and a peroxidase (E.C. 1.11.1.7), and the first dye may be a compound capable of being oxidized by a peroxidase. Thus in this case one of the following cascades of enzyme catalysed chemical reactions may take place:
beta-D-galactofuranoside+beta-galactofuranosidase→galactose+galactose oxidase→H2O2+first dye (red)+Peroxidase→second dye (ox),
wherein the term “(red)” and “(ox)” refers to the reduced and oxidized forms of the same compound.

If the enzyme of interest is a carboxypeptidase A (E.C. 3.4.17.1), the first substrate may be a polypeptide, the other enzymes may be an L-amino acid oxidase (E.C. 1.4.3.2) and a peroxidase (E.C. 1.11.1.7), and the first dye may be a compound capable of being oxidized by a peroxidase. Thus in this case the following cascade of enzyme catalysed chemical reactions may take place:
Polypeptide+carboxypeptidase A→amino acid+L-amino acid oxidase→H2O2+first dye (red)+Peroxidase→second dye (ox),
wherein the term “(red)” and “(ox)” refers to the reduced and oxidized forms of the same compound.

For testing or screening for the activity of an oxidase the enzyme of interest may be an oxidase, e.g. one of those described above, the first substrate may be any compound which is a substrate for the particular oxidase of choice, the other enzyme may be a peroxidase (E.C. 1.1 1.1.7) and the first dye may be a compound capable of being oxidized by the peroxidase. For example if the oxidase is a glucose oxidase and the first substrate is beta-D-glucose the following cascade of enzyme catalysed chemical reactions may take place:
Glucose+Glucose Oxidase→H2O2+first dye (red)+Peroxidase→second dye (ox),
wherein the term “(red)” and “(ox)” refers to the reduced and oxidized forms of the same compound. Similar reactions may take place when the enzyme of interest is another oxidase and the first substrate is a substrate for the oxidase.
Dye

The first dye of the present invention should be a substrate for an enzyme for which a second dye is a product of the chemical reaction between the enzyme and the first dye and wherein the second dye has a different colour than the first dye. Said enzyme may be an other enzyme or an enzyme of interest, depending on for which method of the present invention the first dye is used for.

Schematically shown the first dye should be capable of being involved in the following chemical reaction:
First dye+enzyme→second dye

The term “colour” refers in the context of the present invention either to colours which are visible to the human eye (i.e. electromagnetic radiation with a wavelength of between 400 and 700 nm) or to the emission of a fluorescent signal by the first and/or second dye. If white light is spread out by a prism, we can see that it is composed of different colours. Each colour corresponds to a different wavelength. The colour of a compound depends on the wavelength of light which it absorbs. If a compound does not absorb any visible light it will be colourless. If a compound absorbs light humans perceive the complementary colour, because the light which reaches our eyes is missing the wavelengths which have been absorbed. The relation between some wavelengths of visible light and colour are:

| Wavelength of light, nm | Colour | Complementary colour |
|---|---|---|
| 400-430 | Violet | Green-yellow |
| 430-480 | Blue | Yellow |
| 480-490 | Green-blue | Orange |
| 490-510 | Blue-green | Red |
| 510-530 | Green | Purple |
| 530-570 | Yellow-green | Violet |
| 570-580 | Yellow | Blue |
| 580-600 | Orange | Green-blue |
| 600-680 | Red | Blue-green |

Thus the term “different colour” refers in this context to that the first and second dye absorbs different wavelengths of visible light or that the first dye does not absorb any light within the visible area (i.e. it is colour-less), while the second dye does absorb light of the visible area or vice versa.

The first and/or second dye may also be a compound capable of emitting a fluorescent signal. Emission of fluorescence is achieved by excitation of the fluorescent compound using a specific electromagnetic radiation wavelength, the specific wavelength depends on the compound. The emission wavelength is different from the excitation wavelength. In this context the term “different colour” in reference to the first and second dye refers then to the ability of the first dye to emit fluorescent light, while the second dye does not or vice versa, or that the intensity of the fluorescent light emitted from the first dye is either increased or decreased as compared to the intensity of the fluorescent light emitted from the second dye.

In a particular embodiment of the present invention the enzyme which reacts with the first dye is a peroxidase, i.e. the first dye is a compound capable of being oxidized by a peroxidase (E.C. 1.11.1.7) to a second dye. Thus the first dye is a reduced form of a compound and the second dye is the oxidized form of the compound.

Examples of first dyes which are capable of being oxidized to a second dye in the presence of a peroxidase (and hydrogen peroxide) include, but are not limited to: Dyes of the class Triphenyl methane; e.g. Crystalviolet, Malachite green or bromophenol blue, or dyes of Azo class; e.g. Methyl orange or Congo red, or Phenol based dyes; Phenol red, or Polymeric dyes; e.g. Poly R478; Anthraquinone-based; e.g. Remazol Brilliant Blue, or cationic dyes; e.g. ruthenium red, or anionic dyes; e.g. eosin.

Above examples of first dyes are all dyes where the change in colour from the first to the second dye is change which is detectable by visible light. However, as described above the change in colour from the first to the second dye may also relate to a fluorescent signal. In this case if the first dye should be a substrate for a peroxidase the first dye may be a non-oxidized form of a compound which upon oxidation by a peroxidase emits a fluorescent signal with an intensity which is either increased or decreased as compared to the emission wavelength of the non-oxidised form (first dye). The difference in the emission of the oxidised form (second dye) may then be detected in a background of the non-oxidized form (first dye). Examples of first dyes which comprise this ability include but are not limited to 10-Acetyl-3,7-dihydroxyphenoxazine (Amplex red™ from Molecular Probes, USA) In the presence of horseradish peroxidase (HRP), the Amplex Red reagent reacts in a 1:1 stoichiometry with H2O2 to produce highly fluorescent resorufin (Optimal Excitation wavelength is nm 544 and optimal emission wavelength is nm 590).

Polymer

The polymer of the present invention may be any polymer capable of binding the second dye. An advantage of using a polymer capable of binding the second dye is that diffusion of the second dye in the solid media is reduced, thereby making it easier to identify an enzyme of interest or a host cell expressing an enzyme of interest. This may be of particular importance if the border of the colour-zone surrounding the enzyme of interest or the host cell is difficult to define.

Examples of suitable polymers include but are not limited to: carboxymethyl-cellulose (CMC), chitin, pectate, pectin, starch e.g. potato starch, locust bean gum and ghatti gum. Typically the choice of polymer depends on the identity of the second dye. Thus for example if the first dye is Brilliant Blue or Congo Red the polymer may in particular be CMC, chitin, pectate, pectin or starch.

If the first dye is Ruthenium Red the polymer may in particular be pectate or pectin. If the first dye is Eosin the polymer may in particular be chitin or chitosan.

Host Cells

The enzyme of interest may be expressed by any host cell or the library of polypeptides may be expressed by a library of host cells. The host cell may a prokaryotic or eukaryotic cell, for example it may be a bacterium, fungus, such as yeast or a filamentous fungus, mammalian, plant or an insect cell. The enzyme may be native or foreign to the host cell, i.e. it may be expressed naturally by the host cell or it may be an enzyme the host cells does not express naturally. The host cell may have been manipulated to express the enzyme of interest/library of polypeptides, e.g. by introducing a nucleic acid sequence encoding the enzyme of interest/library of polypeptides into the host cell, or the host cell may be cell which naturally expresses the enzyme of interest/library of polypeptides.

Examples of bacterial host cells include gram-positive bacteria such as a strain of Bacillus, e.g. strains of B. subtilis, B. licheniformis, B. lentus, B. brevis, B. stearothermophilus, B. alkalophilus, B. amyloliquefaciens, B. coagulans, B. circulans, B. lautus, B. megaterium or B. thuringiensis, or strains of Streptomyces, such as S. lividans or S. murinus, or gram-negative bacteria such as Escherichia coli or Pseudomonas sp.

Transformation of bacteria may be effected by protoplast transformation, electroporation, conjugation, or by using competent cells in a manner known per se (cf. Sambrook et al., supra).

If the enzyme of interest is expressed by gram-positive bacteria such as a Bacillus or Streptomyces strain, the enzyme/polypeptide may be retained in the cytoplasm, or it may be directed to the extracellular medium by a bacterial secretion sequence.

If the enzyme of interest/library of polypeptides is expressed by a gram-negative bacteria such as E. coli, the enzyme/polypeptide may be retained in the cytoplasm, typically as insoluble granules (known as inclusion bodies), or it may be directed to the periplasmic space by a bacterial secretion sequence. In the former case, the cells may typically be lysed. In the latter case, the enzyme/polypeptide may be released from the periplasmic space by disrupting the cells, e.g. by sonication or osmotic shock, to release the contents of the periplasmic space.

Examples of host yeast cells include cells of a species of Candida, e.g. C. maltose, or Kluyveromyces, e.g. K. lactis, K. fragilis, or Saccharomyces, e.g. S. carlsbergensis, S. cerevisiae, S. diastaticus, S. douglasii, S. kluyveri, S. norbensis or S. oviformis, or Schizosaccharomyces, e.g. S. pombe, or Pichia, e.g. P. pastoris, P. guillermondii or P. methanolio, or Hansenula, e.g. H. polymorpha, or Yarrowia, e.g. Y. lipolytica or Ustilgo maylis (cf. Gleeson et al., J. Gen. Microbiol. 132, 1986, pp. 3459-3465; U.S. Pat. No. 4,882,279 and U.S. Pat. No. 4,879,231). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacte-riol, Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981). Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; or Hinnen et al., 1978, Proceedings of the National Academy of Sciences USA 75:1920.

Examples of filamentous fungal cells include filamentous forms of the subdivision Eumycota and Oomycota (as defined by Hawksworth et al., 1995, supra), in particular it may a cell of a species of Acremonium, such as A. chrysogenum, or Aspergillus, such as A. awamori, A. foetidus, A. japonicus, A. niger, A. nidulans or A. oryzae, or Fusarium, such as F. bactridioides, F. cerealis, F. crookwellense, F. culmorum, F. graminearum, F. graminum, F. heterosporum, F. negundi, F. reticulatum, F. roseum, F. sambucinum, F. sarcochroum, F. sulphureum, F. trichothecioides or F. oxysporum, Humicola, such as H. insolens or H. lanuginose, or Mucor, such as M. miehei, or Myceliophthora, such as M. thermophilum, or Neurospora, such as N. crassa, or Penicillium, such as P. purpurogenum, or Thielavia, such as T. terrestris, or Tolypocladium, or Trichoderma, such as T. harzianum, T. koningii, T. Iongibrachiatum, T. reesei or T. viride, or a teleomorph or synonym thereof. The use of Aspergillus spp. for the expression of proteins is described in, e.g., EP 272 277, EP 230 023.

Examples of insect cells include a Lepidoptera cell line, such as Spodoptera frugiperda cells or Trichoplusia ni cells (cf. U.S. Pat. No. 5,077,214). Culture conditions may suitably be as described in WO 89/01029 or WO 89/01028.Transformation of insect cells and production of heterologous polypeptides therein may be performed as described in U.S. Pat. No. 4,745,051; U.S. Pat. No. 4,775,624; U.S. Pat. No. 4,879,236; U.S. Pat. No. 5,155,037; U.S. Pat. No. 5,162,222; EP 397,485.

Examples of mammalian cells include Chinese hamster ovary (CHO) cells, HeLa cells, baby hamster kidney (BHK) cells, COS cells, or any number of other immortalized cell lines available, e.g., from the American Type Culture Collection. Methods of transfecting mammalian cells and expressing nucleic acid sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845. Methods for transfecting mammalian cells are well known to a person skilled in the art and include transfection by direct uptake using the calcium phosphate precipitation method of Graham and Van der Eb (1978, Virology 52:546).

Cultivation of Host Cells

In the method of the present invention the enzyme of interest/library of polypeptides is contacted with a solid media and the host cell/library of host cells is cultivated on or in a solid media. In the context of the present invention the term “solid media” refers to the basic state of matter as a solid and to compounds which are solid at 4-70° C., such as between 4-60° C., or at 4-50° C., or at 4-40° C., or at 4-30° C., or at 4-20° C., or at 10-60° C., or at 10-50° C., or at 10-40° C., or at 10-30° C., or at 10-20° C., or 15-40° C., or at 15-30° C., or at 15-25° C., or at 18-23° C. 1 atm. In the context of solid media for these include, but are not limited to, media which are solidified using a solidifying agent such as agar. Other examples of compounds which may be used a solid media include, but are not limited to agar, agarose, pectate, pectin or gelatine, however this is not an exhaustive list and other examples of solid media are well-known to a person skilled in the art.

In one embodiment of the present invention the solid media may be a conventional plate, e.g. a conventional agar plate or a plate comprising another solid media or a plate comprising a combination of different solid media.

In another embodiment of the present invention the solid media may be in the form of a bead, e.g. a bead of agar or another solid media such as one of those described above. For example the enzyme of interest/library of polypeptides or host cells/library of host cells of the present invention may be encapsulated in a small sphere in a way that allows the host cells to multiply and form a colony within its respective sphere or bead. In this way the individual host cells and polypeptides are compartmentalisation. Methods for performing the encapsulation within beads of appropriate size and homogeneity have been developed (Nir et al., Appl. Environ. Microbiol. 56:2870-2875, 1990). This example is based on low-melting-agarose as the gelling/solidifying agent but is not limited to this, other gelling agents such as guar gum, pectate/pectin may also be used.

Measuring the Colour of the Second Dye

Measuring the colour of the second dye may be detected by any means, e.g. visually or automatic. The method of choice typically depends on the choice of solid media, e.g. on whether it is a plate or a bead.

For example if the solid media is a conventional agar plate an enzyme of interest may e.g. be detected by the presence of a zone or halo surrounding a host cell or a hole in the agar where an enzyme of interest/library of polypeptides has been added, which is of a different colour than the rest of the solid media, as the presence of the enzyme of interest results, as described above, in a conversion of the first dye into a second dye which is of a different colour than the first dye. The presence of such zones or halos surrounding a host cell expressing an enzyme of interest/library of polypeptides or a hole with an enzyme of interest/library of polypeptides peptides may then be used to identify an enzyme of interest. Said identification of the zones or halos may be performed by visual or automatic inspection of the agar plate.

The contrast between the colour of the first and the second dye may be enhanced by using higher amounts (e.g. higher concentration) of the first dye or as described above by the presence of a polymer capable of binding the second dye and thereby reduce diffusion of the second dye in the solid media.

If the enzyme of interest/library of polypeptides or the host cell/library of host cells has been cultivated in a bead of solid media, then the presence of the enzyme of interest or a host cell expressing an enzyme of interest in a bead may generally cause said bead to change to a different colour than the other beads, as the presence of the enzyme of interest results in a conversion of the first dye to a second. Identification of beads with a changed colour may be performed visually or in particular it may be performed automatically by e.g. the use of a flow cytometer, such as a FACS (Fluorescence-Activated Cell Sorter).

Materials and Methods

Materials

Enzymes

HRP (Horseradish Peroxidase): #P8125, Sigma-Aldrich, which is a peroxidase obtained from Horseradish

GOX (Glucose Oxidase): #G6125, Sigma-Aldrich, which is a glucose oxidase obtained from Aspergillus niger

SC-Blue-Pox-agar.

In 10 L container mix:

| | | |
|---|---|---|
| Agar (#101614, Merck) | 200 | g |
| Yeast Nitrogen W/O aminoacids (#51484, FLUKA) | 75 | g |
| Succinic acid (#S7501, Sigma-Aldrich) | 113 | g |
| NaOH (#6498, Merck) | 68 | g |
| Casamino acids (#0288, DIFCO) | 56 | g |
| L-Tryptophan (#8374, Merck) | 1 | g |
| MilliPore H2O | to 8000 | mL |
| Sterilize by autoclavation. | | |
| Mix to total 10 L | | |

After sterilizing the agar was kept at 60° C. and the following was added: 800 mL 50% Fructose and 100 mL 50% Maltose (maltose and fructose were sterile filtered through 0.2 microm sterilefilter and both solutions were heated to 60° C. before use).

Thereafter 200 mL of 60° C. MilliPore water containing 4 g Remazol Brilliantblue (#R8001 Sigma-Aldrich) was added and the solution was sterile-filtered. Then 1000 mL of 60° C. Millipore water with 50 g CMC (Sodium carboxymethylcellulose, #21900, FLUKA, Sigma-Aldrich) was added. The CMC solution was heated on a magnetic stirrer until it was solubilised, before use the CMC was autoclaved.

Just before pouring plates the following was added:
240 microL HRP (5000 U/mL Horseradish peroxidase (HRP) (Dilution of #P8125, Sigma-Aldrich),
5 mL GOX (2500 U/mL Glucose Oxidase (GOX) (Dilution of #G6125, Sigma-Aldrich) and
4 mL AMP 250mg/mL Ampicillin (#A9518, Sigma-Aldrich)

### EXAMPLES

### Example 1

Testing Expression of AMG by Yeast on a SC-Blue-Pox-Agar-Plate

A derivative of the yeast Saccharomyces cerevisiae ATCC 26109 strain (the derivative has been disrupted in the Ura3 gene using a 5-FOA selection, making it Uracil dependent) was transformed with a plasmid containing the gene of the Talaromyces emersonii amyloglycosidase (AMG) under transcriptional control of the Triose Phosphate Isomerase promoter. The plasmid is a derivative of pYES2 (Invitrogen) and encodes a 2my origen of replication for propagation in yeast and the Ura3 gene (for positive selection of plasmid containing yeast clones on Uracil free plates). Furthermore the plasmid contains E.coli origen of replication and ampicillin resistance gene derived from pUC19 plasmid. (The plasmid was constructed essentially as described in the Material and Methods section of patent WO200104273). The signal peptide of the Talaromyces emersonii AMG directs the enzyme to the exterior of the cell.

The transformed cells were plated onto a SC-Blue-Pox-agar-plate (see recipe below).

One positive clone expressing AMG was identified and kept as the positive control strain: AMG+.

The positive expression of AMG from this strain was verified by incubating the yeast strain in 10 ml YPD media at 30° C. for 4 days and testing the supernatant in the AMG assay essentially as described in the Material and Methods section of patent WO200104273.

A negative control of no AMG expression was the Uracil dependent the yeast Saccharomyces cerevisiae ATCC 26109 strain. Whenever this strain (called AMG−) was tested, Uracil was included in liquid and solid media (final concentration of 20 mg/l of Uracil). AMG+ and AMG− strains were suspended in water and a small amount of further diluted cells was spread out on SC-Blue-Pox-agar-plate (with and without Uracil respectively for each strain) after 2-3 days incubation at 30 degrees Celsius. AMG− strain had no orange clearing zones around colonies and AMG+ strain expressing AMG were detected visually by the presence of a clear orange halo surrounding the clone as the following reaction between the AMG and the compounds (maltose, Glucose Oxidase, Horseradish Peroxidase and Brilliant Blue) present in the agar plate takes place:
AMG+maltose glucose+Glucose Oxidase H₂O₂+Horseradish Peroxidase+Brilliant Blue→Orange compound
Results:

After incubation for 2-3 days orange halos were present around colonies of the AMG+ strain and not around the AMG− strain, which indicated that AMG+ colonies expressed active AMG. No orange halos could be seen around AMG− colonies even after 7 days of incubation.

### Example 2

Testing the Activity of a Lactase on a SC-Blue-Pox-Lactose-Agar-Plate

A SC-Blue-Pox-Lactose-agar was prepared similarly to the SC-Blue-Pox-agar described in the “Materials” section with the following changes: MilliPore H2O was added to 8300 mL and instead of adding 800 mL 50% Fructose and 100 mL 50% Maltose to the agar 600 mL 50% Lactose was added. Furthermore, the 4 mL Ampicillin was substituted with 10 mL Kanamycin sulfate (10 mg/mL).

After pouring agar into Petri-plates (the agar-layer was approximately 1 cm thick) 4 mm wholes were punched into the agar. Lactase (from Aspergillus oryzae with 17.4 U/mg from Sigma #96049) was diluted to 1 mg/ml, 0.5 mg/ml and 0.1 mg/ml in water. 15 μl of each Lactase concentration were added to the wholes in the agar. The plates were incubated 2-3 hours at 37° C. After this incubation the appearance of orange haloes around the agar wholes, were evidence of the ability of the Lactase to convert Lactose into Glucose and Galactose and ultimately resulting in the oxidation of Brilliant Blue from a blue compound to an orange compound as described below:
Lactase+lactose→galactose+glucose+Glucose Oxidase→H2O2+Horseradish Peroxidase+Brilliant Blue Orange compound

### Example 3

Screening Yeast Host Cells Comprising a Diversified Library of AMG for AMG Activity on SC-Blue-Pox-Agar-Plates

The method described in example 1 was repeated with the only exception that it was used to screen a diversified library of the Talaromyces emersonii amyloglycosidase (AMG) gene which had been introduced into a derivative of the yeast Saccharomyces cerevisiae ATCC 26109 strain (the derivative has been disrupted in the Ura3 gene using a 5-FOA selection, making it Uracil dependent). The transformed yeast cells were plated on SC-Blue-Pox-agar-plates according to example 1. Clones expressing active AMG were identified by the presence of an orange halo surrounding the colony. Positive clones were transferred (using tooth-picks) form plate to micro well plates with SC-Ura medie for growth at 30° C. for 4-5 days. Expression of active AMG by a clone was verified by an AMG assay, see example 4. An advantage of using this assay to screen a diversified library is that such libraries often contain a substantially high proportion of inactive enzyme variants. It is therefore desirable to enrich the proportion of active clones before initiating a high-through-put screen based on e.g. microtiter plates or other encapsulation techniques.

### Example 4

Screening the Supernatants of Yeast Host Cells Expressing a Diversified Library of AMG

Expression of a diversified library of the Talaromyces emersonii amyloglycosidase (AMG) in the yeast Saccharomyces cerevisiae ATCC 26109A was prepared as described in example 3.

However, in the present example it was the supernatant from yeast cells expressing the library which was tested on the Blue-Pox plates rather than plating the yeast cells themselves onto the plates.

Blue-Pox plates were prepared by mixing insoluble starch with molten agar typically at a 0.5-5% concentration (w/v) in a buffer of choice. The molten agar was then mixed with Horse Radish Peroxidase, Glucose oxidase, Brilliant Blue, CMC (carboxy-methyl-cellulose) to prepare SC-Blue-Pox-agar as described above and poured onto petridish plates. Thus, the plates in this example use insoluble starch as substrate for the AMG rather than the Maltose used in example 1.

Following solidification of the agar small cavities/holes was punctuated into the surface to form reservoirs. Small volumes of culture supernatants containing the libraries of interest were thereafter placed in these holes whereby the enzyme present in the culture supernatant could contact the insoluble substrate along the entire side of the punctuated hole. Activity was visualised as halos forming around the punctuated holes.

The ability to trap an otherwise insoluble substrate in a solidified agar or agarose (or other solidifiable substance such as alginate) allows the Blue-pox assay to be performed on substrates that otherwise are difficult to screen and at conditions that otherwise could be incompatible with the expression host cell.

1-22. (canceled)

23. A method for testing an enzyme of interest or screening a library of polypeptides for an enzyme of interest comprising measuring the colour of a second dye, wherein the enzyme of interest or library of polypeptides has been contacted with a solid media in the presence of a first substrate, one or more other enzymes and a first dye, and wherein a product of the chemical reaction between an enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye.

24. The method according to claim 23, wherein a polymer capable of binding the second dye is also present.

25. The method according to claim 23, wherein the other enzymes comprise a peroxidase (E.C. 1.11.1.7).

26. The method according to claim 23, wherein the other enzymes further comprise an enzyme capable of producing hydrogen peroxide upon reaction with its substrate, e.g. a glucose oxidase (E.C. 1.1.3.4), a cellobiose oxidase (E.C. 1.1.3.25), an alcohol oxidase (E.C. 1.1.3.13), a galactose oxidase (E.C. 1.1.3.9) or a L-amino acid oxidase (E.C.1.4.3.2).

27. The method according to claim 23, wherein the enzyme of interest is selected from the group consisting of: a glucoamylase (E.C. 3.2.1.3), a beta-glucosidase (E.C. 3.2.1.21), a pectinesterase (E.C. 3.1.1.11), a alpha-galactosidase (E.C. 3.2.1.22), a cellulose 1,4-beta-cellobiosidase (E.C. 3.2.1.91), a lactase (E.C.3.2.1.108), a beta-galactofuranosidase and a carboxypeptidase A (E.C. 3.4.17.1).

28. The method according to claim 23, wherein the other enzymes further comprise a beta-glucosidase (E.C. 3.2.1.21).

29. The method according to claim 23, wherein the enzyme of interest is a cellulase (E.C.3.2.1.4) or a cellulose 1,4-beta-cellobiosidase (E.C. 3.2.1.91).

30. The method according to claim 23, wherein the enzyme of interest is an enzyme for which a product of the chemical reaction between the enzyme of interest and a first substrate is hydrogen peroxide.

31. The method according to claim 30, wherein the enzyme of interest is selected from the group consisting of: a glucose oxidase (E.C. 1.1.3.4), a cellobiose oxidase (E.C. 1.1.3.25), an alcohol oxidase (E.C. 1.1.3.13), a galactose oxidase (E.C. 1.1.3.9) and a L-amino acid oxidase (E.C.1.4.3.2).

32. A method for testing a host cell or screening a library of host cells for expression of an enzyme of interest comprising measuring the colour of a second dye, wherein the host cell or library of host cells has been cultivated on or in a solid media in the presence of a first substrate, one or more other enzymes and a first dye, and wherein a product of the chemical reaction between the enzyme of interest and the first substrate is a substrate for one of the other enzymes, and wherein the first dye is a substrate for one of the other enzymes, and wherein the product of the chemical reaction between the first dye and one of the other enzyme is a second dye, and wherein the colour of the first dye is different from the colour of the second dye.

33. A method according to claim 32, wherein the method comprises the following steps:
a) cultivating a host cell expressing the enzyme of interest or a library of host cells expressing a library of polypeptides on or in a solid media in the presence of a first substrate, one or more other enzymes and a first dye, wherein a product of the chemical reaction between the enzyme of interest and the first substrate is a substrate for one of the other enzymes, and wherein the first dye is a substrate for one of the other enzymes, and wherein the product of the chemical reaction between the first dye and one of the other enzymes is a second dye, and wherein the colour of the first dye is different from the colour of the second dye.
b) measuring the colour of the second dye.

34. The method according to any of claim 32, wherein a polymer capable of binding the second dye is also present.

35. The method according to claim 32, wherein the polymer is carboxy methyl cellulose (CMC), chitin, chitosan, pectate, pectin or starch.

36. The method according to claim 32, wherein the other enzymes comprise a peroxidase (E.C. 1.11.1.7).

37. The method according to claim 32, wherein the other enzymes further comprise an enzyme capable of producing hydrogen peroxide upon reaction with its substrate, e.g. a glucose oxidase (E.C. 1.1.3.4), a cellobiose oxidase (E.C. 1.1.3.25), an alcohol oxidase (E.C. 1.1.3.13), a galactose oxidase (E.C. 1.1.3.9) or a L-amino acid oxidase (E.C.1.4.3.2).

38. The method according to claim 32, wherein the enzyme of interest is selected from the group consisting of: a glucoamylase (E.C. 3.2.1.3), a beta-glucosidase (E.C. 3.2.1.21), a pectinesterase (E.C. 3.1.1.11), a alpha-galactosidase (E.C. 3.2.1.22), a cellulose 1,4-beta-cellobiosidase (E.C. 3.2.1.91), a lactase (E.C.3.2.1.108), a beta-galactofuranosidase and a carboxypeptidase A (E.C. 3.4.17.1).

39. The method according to claim 32, wherein the other enzymes further comprise a beta-glucosidase (E.C. 3.2.1.21).

40. The method according to claim 32, wherein the enzyme of interest is a cellulase (E.C.3.2.1.4) or a cellulose 1,4-beta-cellobiosidase (E.C. 3.2.1.91).

41. The method according to claim 32, wherein the enzyme of interest is an enzyme for which a product of the chemical reaction between the enzyme of interest and a first substrate is hydrogen peroxide.

42. The method according to claim 32, wherein the enzyme of interest is selected from the group consisting of: a glucose oxidase (E.C. 1.1.3.4), a cellobiose oxidase (E.C. 1.1.3.25), an alcohol oxidase (E.C. 1.1.3.13), a galactose oxidase (E.C. 1.1.3.9) and a L-amino acid oxidase (E.C.1.4.3.2).
